(19) United States
(12) Patent Application Publication
(10) Pub. No.: US 2022/0110664 A1
(43) Pub. Date: Apr. 14, 2022

(54) TWO-PIECE POLYAXIAL TITANIUM HAMMERTOE DEVICE

(71) Applicants: Thomas Zink, San Antonio, TX (US); Matthew Jackman, Sherman, TX (US); Erin England, San Antonio, TX (US)

(72) Inventors: Thomas Zink, San Antonio, TX (US); Matthew Jackman, Sherman, TX (US); Erin England, San Antonio, TX (US)

(21) Appl. No.: 17/496,935

(22) Filed: Oct. 8, 2021

Related U.S. Application Data
(60) Provisional application No. 63/089,577, filed on Oct. 9, 2020.

(51) Int. Cl. A61B 17/72; A61F 2/42

(52) U.S. Cl. CPC A61B 17/7291; A61B 17/7225; A61B 2017/00526; A61F 2/42; A61B 17/7233

(57) ABSTRACT

A two-piece hammertoe compression device is disclosed for the correction of a hammer toe deformity. The two-piece hammertoe compression device provides polyaxial motion between the device components allowing angulation of the joint for fusion. Further, the two-piece hammertoe compression device includes a distal component and a proximal component secured together. The distal component has an externally-threaded anchor with an embedded socket component. The proximal component includes an internally and externally-threaded anchor. Additionally, the embedded socket component has a ball insert component to allow for polyaxial movement and variable angle fixation at the joint.

### CROSS-REFERENCE TO RELATED APPLICATION

The present application claims priority to, and the benefit of, U.S. Provisional Application No. 63/089,577, which was filed on Oct. 9, 2020, and is incorporated herein by reference in its entirety.

### BACKGROUND OF THE INVENTION

The present invention relates generally to a medical device for the foot. More particularly, the present disclosure relates to a two-piece polyaxial hammertoe compression device which corrects a hammertoe deformity with polyaxial motion between the two or separate device components, allowing angulation of the joint for fusion.

A hammertoe is a deformity of the proximal inter-phalangeal joint of the second, third, fourth, or fifth toe causing the toe to be permanently bent and giving the toe(s) the resemblance of a hammer. Initially, hammertoes are flexible and may be corrected with simple measures but, if left untreated. the condition may become permanent and hammertoes may require surgical intervention for correction. Persons with a hammertoe may also have corns or calluses on the top of the middle joint of the toe or on the tip of the toe, due to the rubbing of the toe against the interior of the shoe, and may feel pain in their toes or feet and have difficulty finding comfortable shoes.

Hammertoe deformities are commonly corrected with fusion via inserting screws, wires, or other similar implants in toes to straighten them. Traditional surgical methods generally include the use of Kirschner wires (“K-wires”). K-wires require pins protruding through the end of respective toes due to their temporary nature. As a result, K-wires often lead to pin tract infections, loss of fixation and limitations to a fixed angle. Additional disadvantages occurring with the use of K-wires include migration and breakage of the K-wires, thereby resulting in multiple surgeries. Further, the limitations of a fixed angle provides for very few options for variability of the angle. If the surgeon chooses an implant with an angle, the orientation of the implant at the insertion point is crucial to the correct fixation of the toe(s). Due to the various disadvantages of using K-wires, compression screws or devices are being considered as implant alternatives or other treatment options.

Compression screws or devices may provide a more permanent solution than conventional K-wires, as such implants do not typically require removal and have no protruding ends. Further, with the use of compression screws or devices, a patient may wear normal footwear shortly after the respective surgery and do not have to wait for the removal of the pins associated with placement and setting of K-wires. There are generally two types of known compression device implants: single-unit implants, which comprise a completely or substantially completely threaded body, and do not provide a flexibility to the respective toe in its natural movements, and two-unit implants, which typically have one unit that is anchored into the proximal phalanx, a second unit that is anchored into the distal phalanx and a fitting by which the two units are coupled together. Either or both of the two units may be threaded or have other anchoring structures such as barbs or splaying arms.

Thus, there exists a need for an improved hammertoe compression device that corrects a hammertoe deformity, provides variability of the angle and is not limited by a requirement to have a fixed angle for insertion or placement. The present invention discloses a two-piece hammertoe compression device for the correction of a hammer toe deformity with polyaxial motion between the device components allowing angulation of the joint for fusion. Further, the hammertoe compression device includes a distal component having an externally-threaded anchor with an embedded socket component, and a proximal component with an internally and externally threaded anchor.

Additionally, the embedded socket component has a ball insert component to allow for polyaxial movement and variable angle fixation at the joint. The ball insert component further includes an extended threaded connector component that interlocks with the proximal component to secure the proximal component to the distal component. Thus, the hammertoe compression device provides for a more secure fusion environment, ease in connection of the two components, ability to change compression of the two components to increase compression of the two bones together and for variability of angle fixation at the joint.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one aspect thereof, comprises a two-piece hammertoe compression device for the correction of a hammer toe deformity. The two-piece hammertoe compression device provides polyaxial motion between the device components, thereby allowing angulation of the joint for fusion. Specifically, the two-piece hammertoe compression device has a distal component with an externally-threaded anchor and an embedded socket component, as well as a proximal component having an internally and externally threaded anchor. The distal component and the proximal component are secured together via the embedded socket component during surgery.

In a preferred embodiment, the embedded socket component comprises a ball insert component to allow for polyaxial movement and variable angle fixation at the joint. The ball insert component is movably secured within the embedded socket component, and the shank of the ball insert component extends through the externally threaded anchor and further includes an extended threaded connector component which allows for reversible interlocking with the proximal component to secure the proximal component to the distal component.

In another embodiment, the two-piece hammertoe compression device does not require a set orientation at placement to provide a preferred angle, but instead the ball or flexible joint insert component is movably secured within the embedded socket component, which allows for polyaxial movement and provides for insertion angles ranging from −10° to 10°. Further, the securing of the distal and proximal components within the bone does not allow either component to shift within the bone after placement, allowing for easier and more stable fixation of the components within the bone. Additionally, the threaded interlocking mechanism of the extended threaded connector component and the proximal component allows for easy connection of the two components and the ability to variably change the compression between the two components, increasing the compression of the two bones together which provides for additional treatment options due to individual patient requirements.

In another embodiment, the two-piece hammertoe compression device is manufactured as an additively printed titanium component. Specifically, each component of the two-piece hammertoe compression device, the proximal component and the distal component, are manufactured as an additively printed titanium component which can be tailored to the particular treatment needs of the individual.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed, and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a side view of the two-piece hammertoe compression device disclosing the distal component and the proximal component in accordance with the disclosed specification;

FIG. 2 shows a section view of the two-piece hammertoe compression device disclosing the distal component and the proximal component in accordance with the disclosed invention;

FIG. 3 provides a side view of the embedded socket component of the two-piece hammertoe compression device in accordance with the disclosed structure;

FIG. 4 presents a section view of the embedded socket component of the two-piece hammertoe compression device in accordance with the disclosed description;

FIG. 5 shows a side view of the ball insert component with an extended thread connector component in accordance with the disclosed invention;

FIG. 6 provides a side view of the distal component disclosing the extended thread connector component in accordance with the disclosed specification;

FIG. 7 depicts a section view of the distal component disclosing the embedded socket component and the ball insert component with the extended thread connector component in accordance with the disclosed invention;

FIG. 8 shows a top perspective view of the two-piece hammertoe compression device inserted into a user's joint in accordance with the disclosed specification;

FIG. 9 illustrates a top perspective view of the two-piece hammertoe compression device in use within a user's joint in accordance with the disclosed invention; and

FIG. 10 provides a block diagram showing an exemplary method of inserting the two component hammertoe compression device of the present invention.

### DETAILED DESCRIPTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a two-piece hammertoe compression device for the correction of a hammer toe deformity. The two-piece hammertoe compression device provides polyaxial motion between the device components allowing angulation of the joint for fusion. Further, the two-piece hammertoe compression device includes a distal component comprised of an externally-threaded anchor with an embedded socket or joint component and a proximal component that has an internally and externally-threaded anchor.

Additionally, the embedded socket component comprises a ball insert component to allow for polyaxial movement and variable angle fixation at the joint. The ball insert component is movably-secured within the embedded socket component, and the shank of the ball insert component extends through the externally-threaded cage and further has an extended threaded connector component which allows for reversible interlocking with the proximal component to secure the proximal component to the distal component. Thus, the two-piece hammertoe compression device provides for a more secure fusion environment, ease in connection of the two components, ability to change compression pressure of the two components to increase compression of the two bones together and variability of angle fixation at the joint.

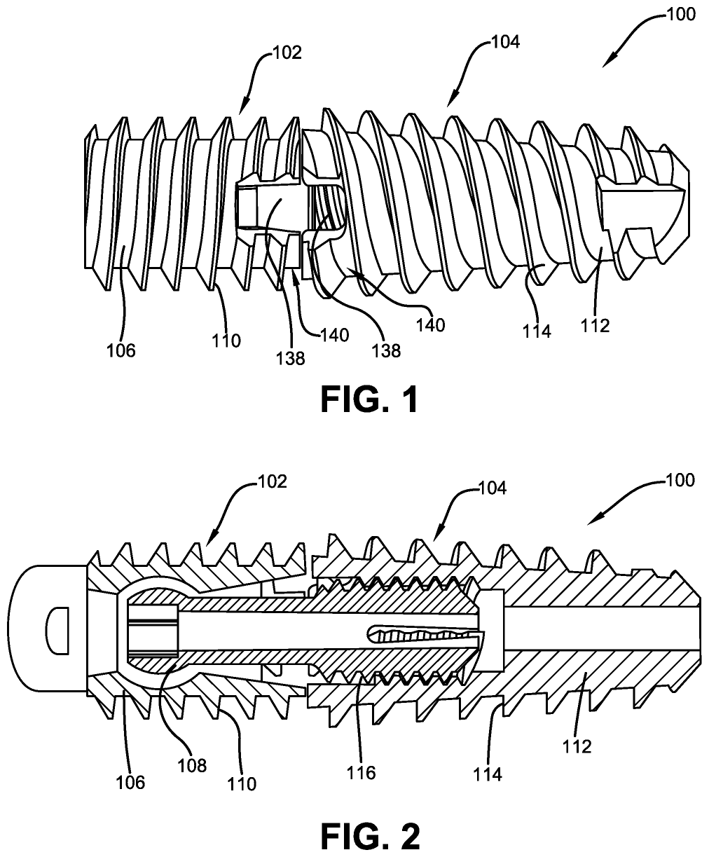

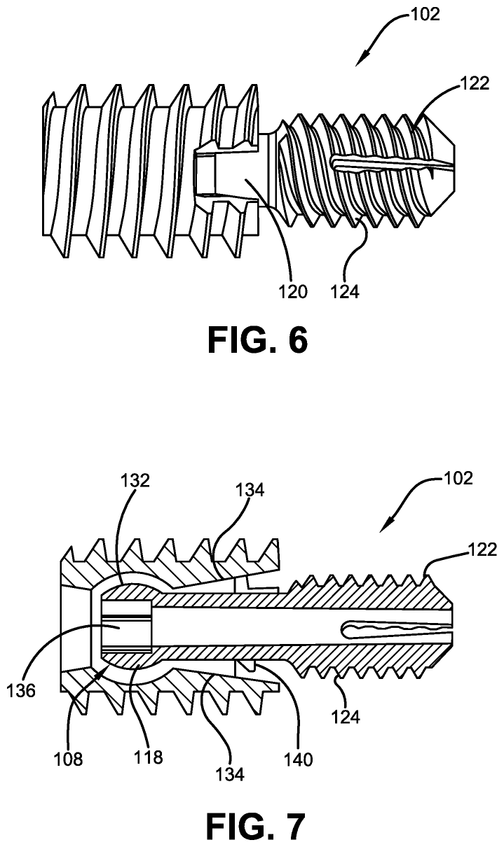

Referring initially to the drawings, FIGS. 1-2 illustrate the two-piece hammertoe compression device 100 which is used for the correction of a hammertoe deformity. Specifically, the two-piece hammertoe compression device 100 comprises a distal component 102 and a proximal component 104 which are secured together. The two-piece hammertoe compression device 100 provides polyaxial motion between the device components 102 and 104 allowing angulation of the joint for fusion. Further, the two-piece hammertoe compression device 100 can be any suitable size, shape and configuration as is needed to meet the needs of different patients without affecting the overall concept of the invention. One of ordinary skill in the art will appreciate that the shape and size of the two-piece hammertoe compression device 100 as shown in FIGS. 1-9 is for illustrative purposes only, and many other shapes and sizes of the two-piece hammertoe compression device 100 are well within the scope of the present disclosure. Although dimensions of the two-piece hammertoe compression device 100 (i.e., length, width, and height) are important design parameters for good performance, the two-piece hammertoe compression device 100 may be any shape or size that ensures optimal performance during use, and may even be customized to fit the exact specifications or measurements of the patient's proximal inter-phalangeal joint.

Additionally, as shown in FIGS. 1-2, the distal component 102 comprises an externally-threaded anchor 106 with an embedded socket component 108. The external threads 110 act to secure and hold the externally-threaded anchor 106 in position within the bone. Further, the distal component 102 comprises a pair of insertion features 138 positioned radially on the outside surface 140. An inserter (not shown) engages with the pair of insertion features 138 to secure the distal component 102 into final position within the bone. The proximal component 104 has an internally and externally-threaded anchor 112. The external threads 114 act to secure the internally and externally-threaded anchor 112 into the bone. Further, the proximal component 104 comprises a pair of insertion features 138 positioned radially on the outside surface 140. An inserter (not shown) engages with the pair of insertion features 138 to secure the proximal component 104 into position within the bone. The distal component 102 and the proximal component 104 are then secured together via the embedded socket component 108 and the internal threads 116 of the proximal component 104 during surgery.

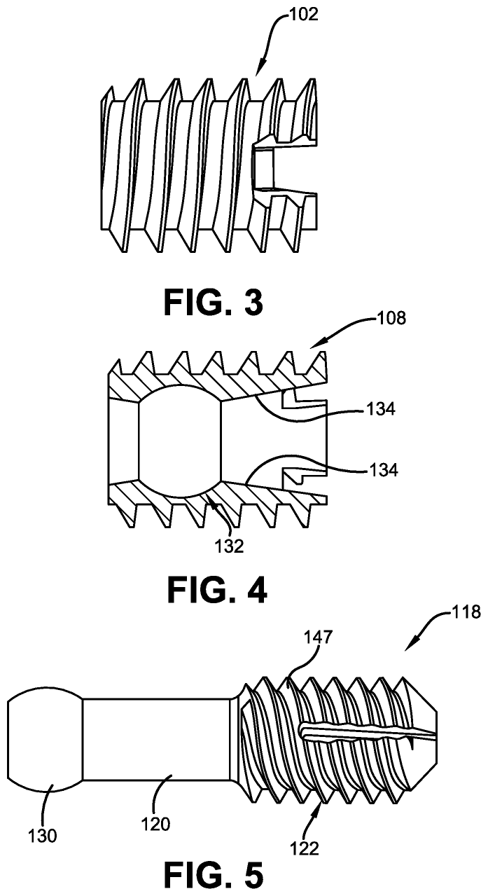

Further, as shown in FIGS. 3-7, the embedded socket component 108 of the distal component 102 includes a ball insert component 118 to allow for polyaxial movement and variable angle fixation at the joint to aid in mimicking normal movement of the joint. The ball insert component 118 is movably-secured within the embedded socket component 108 and the shank 120 of the ball insert component 118 extends through the externally-threaded anchor 106, and further has an extended threaded connector component 122 which allows for reversible interlocking with the proximal component 104 to secure the proximal component 104 to the distal component 102. Specifically, the extended threaded connector component 122 has threads 124 which rotate in a first direction. The proximal component 104 includes internal threads 116 which rotate in a second direction, opposite to the first direction. Thus, the threads 124 of the extended threaded connector component 122 engage with the internal threads 116 of the proximal component 104 to allow for reversible interlocking of the distal component 102 to the proximal component 104.

Additionally, a K-wire (not shown) previously inserted through the middle phalanx and through the distal phalanx and then out the distal tip of the toe, engages with the ball insert component 118 to rotate the extended threaded connector component 122. Specifically, the ball insert component 118 comprises a protruding bulb-like end 130 opposite of the extended threaded connector component 122. The bulb-like end 130 has a rotational feature 136, such as a hex, hexalobe or other suitable shape as is known in the art. The rotational feature 136 engages with the K-wire to rotate the ball insert component 118 which in turn threads the extended threaded connector component 122 of the distal component 102 onto the internal threads 116 of the proximal component 104 to secure the distal component 102 to the proximal component 104.

The K-wire is utilized instead of manual compression to reversibly interlock the distal component 102 to the proximal component 104. The threaded interlocking mechanism allows for easier locking and the ability to change the compression of the two components 102 and 104, which increases the compression of the two bones together. Further, the two-piece hammertoe compression device 100 does not need to be flush with the bone to have compression. This allows for efficient and stable insertion of the two-piece hammertoe compression device 100.

In a preferred embodiment, the two-piece hammertoe compression device 100 does not require a set orientation at surgical placement to provide an angle, but instead the ball insert component 118 is movably-secured within the embedded socket component 108 to allow for polyaxial movement and provide angles from between about −10° to around 10°. Specifically, as shown in FIGS. 4 and 7, the embedded socket component 108 has a socket 132 with tapering sidewalls 134 for variable angles. The protruding bulb-like end 130 of the ball insert component 118 is positioned within the socket 132 of the embedded socket component 108 and is movably secured within the socket 132 via the tapering sidewalls 134 which taper toward the ball 108 which allow the bulb-like end 130 to move and turn within the socket 132, but prevents the bulb-like end 130 from being removed from the socket 132 as the sidewalls sever to lock the bulb-like end 130 within the socket.

Thus, the combination of the embedded socket component 108 and the ball insert component 118 allows for variable angles or pivoting with a single compression device 100 without concern for orientation at surgical placement due to polyaxial movement of the ball insert component 118 within the socket 132. This configuration then allows for a more natural movement of the toe(s). Specifically, the ball insert component 118 within the distal component 102 allows for rotation of the extended threaded connector component 122 for engagement with the proximal component 104. Accordingly, tightening of the two-piece hammertoe compression device 100 does not alter or affect the placement of the distal 102 or proximal 104 components within the bone. Further, the joining of the distal 102 and proximal 104 components does not move either component within the bone, allowing easier and more stable fixation of the components within the bone.

In a preferred embodiment, the two-piece hammertoe compression device 100 is manufactured as an additively-printed titanium component. Specifically, each component of the two-piece hammertoe compression device 100, the proximal component 104 and the distal component 102, are manufactured using additive manufacturing (AM) techniques and grown as one part. For example, the core of the device 100 is produced and then the threaded elements 116 may be built or grown on the surface of the device 100. Additionally, the two-piece hammertoe compression device 100 is additively-printed and able to be manufactured in a variety of sizes as well as to be customizable to fit the exact specifications or measurements of the patient. Further, the two-piece hammertoe compression device 100 is additively-printed with titanium but can be additively-printed with any other suitable metal as is known in the art, such as stainless steel, as long as the metal is medical grade and able to be additively-printed.

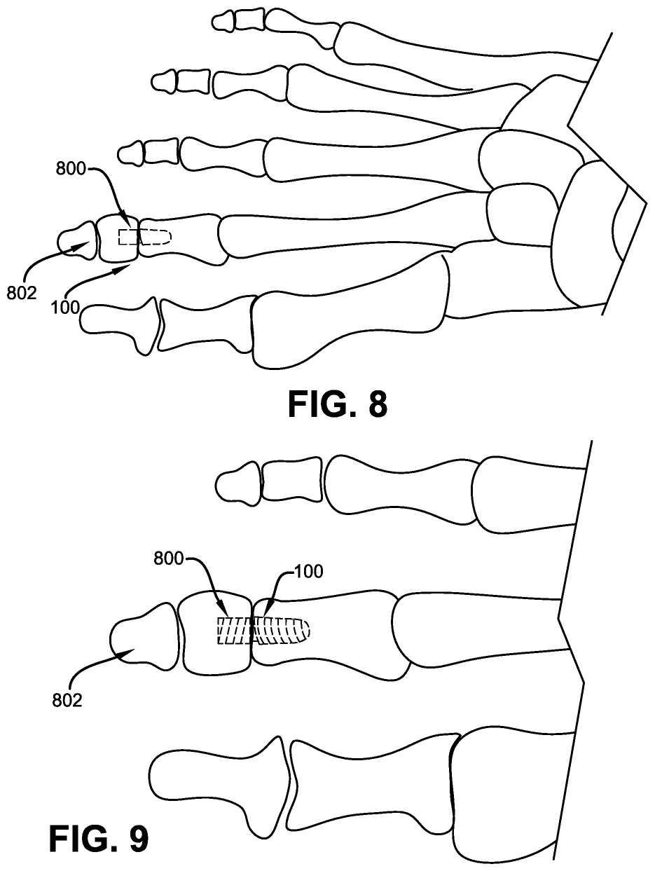

In operation, as shown in FIGS. 8-9, the two-piece hammertoe compression device 100 is applied to the proximal inter-phalangeal joint 800 of the second toe 802. The goal of surgery is to fuse the inter-phalangeal toe joint 800 and make it solidly aligned and immobile. This eliminates much of the pain typically associated with the arthritic joint since there will now be no motion through the arthritic joint. Specifically, an incision is made on top of the toe. Any cartilage is cleared away to allow the two bones to heal or fuse together. To fuse the toe joint, any remnant cartilage on the arthritic joint surface is also removed and the surgeon uses a combination of instruments and tools to shape each bone for a perfect fit, which prepares the underlying bone for fusion.

Once prepared, a K-wire is inserted through the middle phalanx and through the distal phalanx and then out the distal tip of the toe. Then, the distal component 102 is inserted via securing the inserter to the pair of insertion features and threading the distal component 102 into the middle phalanx. The distal component 102 then engages with the K-wire and is secured. Then, the proximal component 104 is inserted via securing the inserter to the pair of insertion features and threading the proximal component 104 into the proximal phalanx. The proximal component 104 is then lined up with the distal component 102.

Further, the proximal component 104 is then secured to the distal component 102 via utilizing the K-wire and threading the extended threaded connector component 122 of the distal component 102 onto the internal threads 116 of the proximal component 104 for reversibly interlocking of the components 102 and 104. As the distal component 102 is threaded onto the proximal component 104, the ball insert component 118 within the distal component 102 allows rotation of the threads 124 for engagement with the proximal component 104. Thus, the tightening of the two-piece hammertoe compression device 100 does not affect placement of the distal 102 or proximal 104 components within the bone, and the components can be set to a desired compression range.

The joint 800 is then positioned in a manner that maximizes the walking ability and maintains acceptable clinical alignment. This is traditionally done with the toe positioned so that it just gently touches the ground in a weight-bearing position. Once the joint is correctly positioned, the distal 102 and proximal 104 components are tightened, compressing the two bones together. Specifically, the components 102 and 104 are tightened until they reach an optimal compression determined by the surgeon to secure the two-piece hammertoe compression device 100 within the proximal inter-phalangeal joint 800.

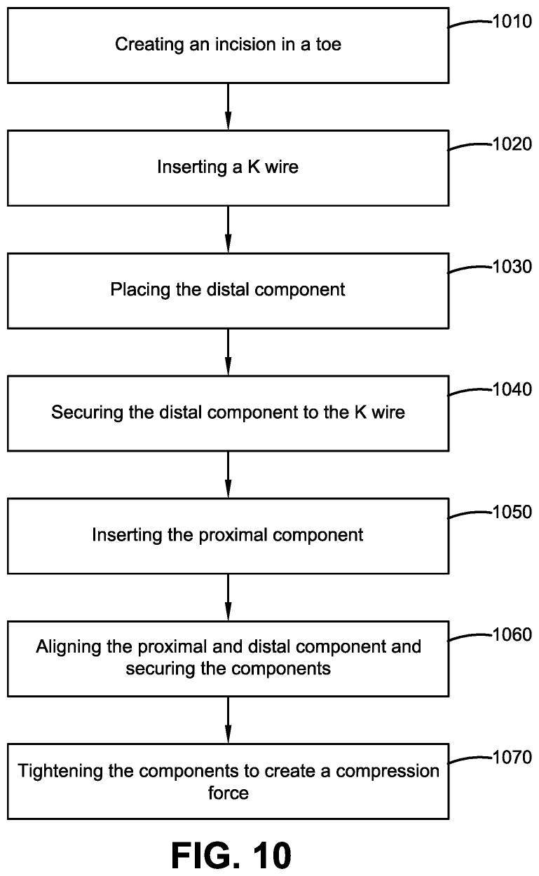

FIG. 10 provides a block diagram for an exemplary method of using a two-component hammertoe correction device. At step 1010, an incision is made in the toe to be treated for the hammertoe condition. Next, at step 1020, a K-wire is inserted into the opening formed by the incision. At step 1030, the proximal portion of the two component system is inserted into the opening created by the incision and secured within the bone by threads positioned on the outer surface of the proximal portion. Step 1040 the proximal portion is secured to the K-wire and at step 1050 the proximal portion is inserted and positioned within the bone. At step 1060 the two components are aligned and at step 1070 a compression force is created by tightening the components.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A two-piece hammertoe compression device for correcting hammertoe deformities, comprising:
a proximal component comprising an internal and external threaded anchor and a pair of insertion features positioned radially on an outside surface of the proximal component;
a distal component comprising an external threaded anchor, an embedded socket component, and a pair of insertion features positioned radially on an outside surface of the distal component; and
wherein the proximal component and the distal component are reversibly interlocked together.

2. The two-piece hammertoe compression device of claim 1, wherein the embedded socket component comprises a ball insert component having a shank with an extended threaded connector component, and a rotational feature.

3. The two-piece hammertoe compression device of claim 2, wherein the extended threaded connector component is secured to the internal threads of the proximal component.

4. The two-piece hammertoe compression device of claim 2, wherein the embedded socket component further includes a socket with tapering sidewalls.

5. The two-piece hammertoe compression device of claim 4, wherein the ball insert component is movably secured within the socket via the tapering sidewalls.

6. The two-piece hammertoe compression device of claim 1 wherein the two-piece hammertoe compression device is manufactured using additive manufacturing (AM) techniques.

7. The two-piece hammertoe compression device of claim 1, wherein the proximal and distal components create a compression force.

8. The two-piece hammertoe compression device as recited in claim 7, wherein the compression force is variable.

9. The two-piece hammertoe compression device as recited in claim 1, further including a K-wire.

10. The two-piece hammertoe compression device as recited in claim 9, wherein at least one of the proximal and distal components are secured to the K-wire.

11. A surgical implant for correcting a hammertoe condition, comprising;
a first component having an outer surface with a plurality of threads extending radially about the surface;
a second component having an outer surface with a plurality of threads extending radially about the surface;
a socket component and a ball insert component sized and configured to fit within the socket component; and
the ball component when positioned in the socket components have an variable fixation angle.

12. The surgical implant as recited in claim 11, wherein the variable fixation angle which ranges from about −10° to about 10°.

13. The surgical implant as recited in claim 11, wherein the socket component has tapering sidewalls in an interior area of the socket component.

14. The surgical implant as recited in claim 13, wherein the tapering sidewalls of the socket component form a locking structure preventing the ball component from being withdrawn from the socket component after insertion.

15. The surgical implant as recited in claim 11, wherein the socket component is contained within the second component.

16. The surgical implant as recited in claim 11, further including a K-wire attached to at least one of the first and second components.

17. The surgical implant as recited in claim 11, wherein the first and second components are made from additive manufacturing.

18. The surgical implant as recited in claim 11, wherein the first and second components are connected to one another and the connection creates a variable compression force.

19. A method of using a two-component hammertoe device, comprising the steps of;
making an incision in a toe;
placing a distal component through the incision and securing the distal component to a bone;
inserting a K-wire;
securing the distal component to the K-wire;
inserting a proximal component and securing the proximal component to a bone;
aligning the proximal component and the distal component; and
securing the proximal component and distal component together to create a compression force.

20. The method as recited in claim 19, wherein one of the proximal component or distal component includes a socket component having a ball component secured within the one and the ball component and socket component creating a variable fixation angle which ranges from about −10° to about 10°.
